(19) United States
(12) Patent Application Publication
(10) Pub. No.: US 2019/0076834 A1
(43) Pub. Date: Mar. 14, 2019

(54) Processes Using Multifunctional Catalysts

(71) Applicant: Novomer, Inc., Boston, MA (US)

(72) Inventor: Sadesh H. Sookraj, Cambridge, MA (US)

(73) Assignee: Novomer, Inc., Boston, MA (US)

(21) Appl. No.: 16/058,716

(22) Filed: Aug. 8, 2018

Related U.S. Application Data
(63) Continuation-in-part of application No. 15/701,410, filed on Sep. 11, 2017.

(51) Int. Cl. B01J 31/22; B01J 31/16

(52) U.S. Cl. CPC B01J 31/22; B01J 31/1616; B01J 2231/10; B01J 2231/321; C07D 303/04; B01J 2531/62; B01J 2531/48; B01J 2531/821; B01J 2531/822; B01J 2531/46

(57) ABSTRACT

The present invention is directed to catalysts and processes for catalyzing two or more chemical reactions with a multifunctional catalyst in a reaction vessel. The processes include steps for introducing one or more reagents to a reaction vessel containing a multifunctional catalyst; contacting the one or more reagents with a first portion of the multifunctional catalyst to produce an intermediate; contacting the intermediate with a second portion of the multifunctional catalyst to produce a product; and removing the product from the reaction vessel. In certain embodiments, the multifunctional catalyst may have a first portion with carbonylation functionality for catalyzing the production of a beta-lactone intermediate from an epoxide reagent and a carbon monoxide reagent. In certain embodiments, the multifunctional catalyst may have a second portion with a functionality suitable for polymerization, co-polymerization, and/or modification of a beta-lactone intermediate. In preferred embodiments, the first portion and second portion are bonded to a heterogenous support.

### CROSS-REFERENCES OF RELATED APPLICATIONS

The present application claims benefit from U.S. application Ser. No. 15/701,410 filed Sep. 11, 2017, which is hereby incorporated by reference in its entirety as if fully restated herein.

### FIELD OF THE INVENTION

The present invention generally is directed to catalysts and processes for catalyzing two or more chemical reactions with a multifunctional catalyst in a reaction vessel. More specifically, the processes comprise catalyzing carbonylation of an epoxide in the presence of carbon monoxide and to form a beta-lactone intermediate and subsequently reacting the beta-lactone intermediate. Advantageously, embodiments of the present invention may catalyze carbonylation, polymerization, copolymerization and/or modification in one reaction vessel.

### BACKGROUND OF THE INVENTION

A catalyst is an atom or molecule which may alter a chemical reaction. Generally, the catalyst may provide an alternative mechanism for a chemical reaction with a different transition state and/or activation energy. A portion of a catalyst, such as an atom, ion, or molecule, may have a particular functionality. For example, one portion of a catalyst may have a Lewis acid functionality but another portion of the catalyst may have a Lewis base functionality. A catalyst having multiple functionalities is termed a multifunctional catalyst for the purposes of this invention.

A catalyst may be characterized as a heterogenous catalyst or a homogenous catalyst depending on whether the catalyst is present in the same phase state as other chemical reagents. A homogenous catalyst will generally be in the same phase state and miscible with other chemical reagents. A heterogenous catalyst will generally be in a different phase state and immiscible with the other chemical reagents.

Certain catalysts, termed carbonylation catalysts for the purposes of this invention, may catalyze the carbonylation of epoxides, aziridines, thiiranes, oxetanes, lactones, lactams, and analogous compounds to produce ring-expanded products. Some conventional carbonylation catalysts comprise a metal carbonyl-Lewis acid catalyst such as those described in U.S. Pat. Nos. 6,852,865, 5,310,948; 7,420,064; and 5,359,081. Many conventional carbonylation catalysts are homogenous catalysts which must be separated from the ring-expanded products for further reaction resulting in extra process steps, manufacturing costs, and overly complex production systems.

There exists need for processes providing for carbonylation and subsequent conversion of a beta-lactone in the same reaction vessel. The present invention satisfies this need by providing for carbonylation and subsequent conversion of a beta-lactone in the same reaction vessel with a multifunctional catalyst.

### SUMMARY OF THE INVENTION

The present invention is directed to processes comprising catalyzing two or more chemical reactions with a multifunctional catalyst in the same reaction vessel. Chemical reagents may be introduced to the reaction vessel to contact and react with a first portion of the multifunctional catalyst having a particular functionality to produce an intermediate molecule. The intermediate molecule may react with a second portion and/or another portion of the multifunctional catalyst having a particular functionality to produce a product and/or another intermediate molecule. Advantageously, the processes of the present invention reduce costs associated with facilities, equipment, safety, and personnel by catalyzing two or more chemical reactions with a multifunctional catalyst in the same reaction vessel.

In preferred embodiments, the processes of the present invention provide for carbonylation. The multifunctional catalyst may have a first portion with a carbonylation functionality. The first portion with the carbonylation functionality may comprise a metal carbonyl-Lewis acid catalyst. In preferred embodiments, an epoxide reagent and a carbon monoxide reagent may be introduced to a reaction vessel containing a multifunctional catalyst with a first portion having a carbonylation functionality. The epoxide reagent and carbon monoxide reagent may react with the first portion having a carbonylation functionality to produce a beta-lactone intermediate. In some embodiments, the beta-lactone intermediate may react with the first portion having a carbonylation functionality to produce a succinic anhydride intermediate and/or a succinic anhydride product wherein the succinic anhydride intermediate may react with a second portion of the multifunctional catalyst and the succinic anhydride product may be removed from the reaction vessel.

In preferred embodiments, the processes of the present invention provide for polymerization. The multifunctional catalyst may have a first portion with a carbonylation functionality and a second portion having ring-opening polymerization functionality. The second portion having ring-opening polymerization functionality may comprise a carboxylate salt of an organic cation, metal-containing complexes, protonated amine, quaternary ammonium salt, guanidinium group, and/or optionally substituted nitrogen-containing heterocycle to name a few. The epoxide reagent and carbon monoxide reagent may be introduced to a reaction vessel and react with the first portion having a carbonylation functionality to produce a beta-lactone intermediate. The beta-lactone may react with the second portion having ring-opening polymerization functionality to produce a polylactone intermediate and/or polylactone product wherein the polylactone intermediate may react with a third portion of the multifunctional catalyst and the polylactone product may be removed from the reaction vessel.

In preferred embodiments, the processes of the present invention provide for co-polymerization and/or modification. The multifunctional catalyst may have a first portion with a carbonylation functionality and one or more other portions with co-polymerization and/or modification functionality. The one or more other portions may comprise condensation polymerization catalysts, oxide catalysts, precious metal catalysts, and/or combinations therein. In preferred embodiments providing for co-polymerization and/or modification, one or more reagents may be introduced to the reaction vessel for reaction with a beta-lactone intermediate. In certain embodiments, the beta-lactone intermediate may be co-polymerized with.

Advantageously, the processes of the present invention provide for the formation of beta-lactones and subsequent conversion of the beta-lactones without the need for multiple catalysts. Some advantages of the present invention are decreased equipment and handling requirements associated with changing catalysts, transferring intermediates to subsequent reaction vessels, and handling beta-lactones.

In a preferred embodiment, a multifunctional catalyst may comprise a substrate and a multifunctional catalyst which is supported on the substrate. The catalyst may have a first portion of carbonylation functionality catalyzing at least one epoxide reagent and carbon monoxide reagent to produce one or more first intermediate. The second portion of the multifunctional catalyst may be designed to convert the one or more first intermediate to a product or a second intermediate.

In another preferred embodiment, the second portion of the multifunctional catalyst may have polymerization functionality polymerizing one or more intermediates to form a polymer.

Optionally in any embodiment, the carbonylation functionality of the multifunctional catalyst may include a carbonylation catalyst.

Optionally in any embodiment, the carbonylation catalyst may comprise a metal carbonyl-Lewis acid catalyst.

Optionally in any embodiment, the substrate may comprise a heterogeneous support.

Optionally in any embodiment, the second portion of the multifunctional catalyst may include a moiety comprising an ionic initiator.

Optionally in any embodiment, the carbonylation functionality of the multifunctional catalyst and the ionic initiator may form a mixture on the substrate.

Optionally in any embodiment, the substrate may have a first zone and a second zone downstream of the first zone. At least about 90 weight percent of the carbonylation functionality of the multifunctional catalyst is located in the first zone and at least about 90 weight percent of the polymerization initiator is located in the second zone.

Optionally in any embodiment, a volume ratio between the first and second zones may be from about 1:10 to about 10:1.

Optionally in any embodiment, heterogeneous support may be selected from the group comprising silica, magnesia, alumina, titania, silica/alumina, pyrogenic silica, high purity silica, zirconia, zincate, carbon, zeolites and mixtures thereof.

Optionally in any embodiment, the at least one epoxide reagent may comprise an ethylene oxide reagent.

Optionally in any embodiment, the one or more intermediates comprises a beta-lactone intermediate.

Optionally in any embodiment, the one or more intermediates includes a succinic anhydride intermediate.

Optionally in any embodiment, the multifunctional catalyst may comprise a third portion of functionality polymerizing acrylic nitrile.

Optionally in any embodiment, the third portion comprises a metal complex bonded to the substrate. The metal complex may comprise a metal chosen from Ti, Cr, Mn, Fe, Ru, Co, Rh, Sm, Re, Ir, Zr, Ni, Pd, Co, Zn, Mg, Al, Ga, Sn, In, Mo, W, for example.

Optionally in any embodiment, the second portion of the multifunctional catalyst includes a moiety comprising a dehydration agent.

Optionally in any embodiment, the dehydration agent is chosen from the group consisting of a phosphorous pentoxide, an organophosphorous compound, a carbodiimide compound, a triazine compound, an organosilicon compound, a transition metal complex, an aluminum complex, and a mixture thereof.

Optionally in any embodiment, the one or more intermediates includes a succinic anhydride intermediate.

Optionally in any embodiment, the second intermediate comprises acrylic acid.

While this disclosure is susceptible to various modifications and alternative forms, specific exemplary embodiments thereof have been shown by way of example in the drawings and have herein been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

### DETAILED DESCRIPTION OF EMBODIMENTS

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The present invention is directed to processes comprising steps for catalyzing two or more chemical reactions with a multifunctional catalyst in a reaction vessel. In certain embodiments, the processes may include a reaction vessel configured so that the processes of the present invention may be performed continuously. In some embodiments, the reaction vessel may be configured as a fixed-bed continuous reactor or fluidized-bed continuous reactor. The reaction vessel comprises mainly metal materials, such as carbon steel and stainless steel, and use of stainless steel is preferable. The processes of the present invention may include reaction vessels having one or more reaction zones, inlets, outlets, heat exchange surfaces, filters, and/or mixers.

In preferred embodiments, the processes comprise the steps: introducing one or more reagents to a reaction vessel; contacting the one or more reagents with a first portion of a multifunctional catalyst to produce an intermediate; contacting the intermediate with a second portion of a multifunctional catalyst to produce a product; and removing the product from the reaction vessel.

In certain preferred embodiments, a feed stream comprising one or more reagents may be introduced to a reaction zone of the reaction vessel containing a multifunctional catalyst. The feed stream may be introduced as a gas, liquid, and/or solid. The feed stream may be introduced at lower than ambient temperature, ambient temperature, or higher than ambient temperature. The feed stream may be introduced at lower than ambient pressure, ambient pressure, or higher than ambient pressure. In some embodiments, it may be preferable to introduce a first reagent to the reaction vessel before introducing a second reagent to the reaction vessel. In certain embodiments, the feed stream may comprise two or more feed streams introduced to the reaction vessel at different phases, temperatures, pressures, and/or times.

In certain preferred embodiments, one or more reagents may diffuse to contact a first portion of the multifunctional catalyst and produce an intermediate. In certain embodiments, one or more reagents may diffuse as a gas to contact the first portion of a solid phase multifunctional catalyst. For example, one or more reagents introduced to a reaction vessel as liquid at ambient temperature may be heated to gas phase and diffuse to contact the first portion of the solid phase multifunctional catalyst. In another example, one or more reagents introduced to a reaction vessel as a gas may diffuse to contact the first portion of the solid phase multifunctional catalyst. In certain embodiments, one or more reagents may diffuse as a liquid to contact the first portion of a solid phase multifunctional catalyst. For example, one or more liquid phase reagents may be introduced to a reaction vessel above a solid phase multifunctional catalyst and diffuse to contact the first portion under the force of gravity.

In certain preferred embodiments, an intermediate may diffuse to contact a second portion of the multifunctional catalyst and produce a product. In certain embodiments, the intermediate may diffuse as a gas to contact the second portion of a solid phase multifunctional catalyst. For example, an intermediate in liquid phase may be heated to gas phase and diffuse to contact the second portion of the solid phase multifunctional catalyst. In certain embodiments, an intermediate may diffuse as a liquid to contact the second portion of a solid phase multifunctional catalyst. For example, an intermediate may diffuse to contact the second portion under the force of gravity. In some embodiments, the intermediate may be cooled from gas phase to liquid phase and diffuse to contact the second portion under the force of gravity.

Preferably, the product may be removed from the reaction vessel in a liquid phase or a gas phase. In preferred embodiments, the multifunctional catalyst may remain in the reaction vessel in a solid phase. In certain preferred embodiments, a product in liquid phase may be heated to gas phase and removed from the reaction vessel. In certain other preferred embodiments, a product in gas phase may be cooled to liquid phase and removed from the reactor. In certain embodiments, the product may be separated from the multifunctional catalyst, impurities, unreacted reagents, or any other undesired materials by phase separation, distillation, ion exchange filtration, and/or molecular sieve filtration.

The one or more reagents include an epoxide reagent and a carbon monoxide reagent. The epoxide reagent and carbon monoxide reagent may have high biobased carbon content wherein Bio-based content=[Bio (Organic) Carbon]/[Total (Organic) Carbon] 100%, as determined by ASTM D6866 (Standard Test Methods for Determining the Bio-based Content of Solid, Liquid, and Gaseous Samples Using Radiocarbon Analysis). The epoxide reagent and carbon monoxide reagent with high biobased carbon content may be produced, from biologically sourced, renewable, recycled, and/or sustainable sources of carbon such as bio-mass derived carbon, carbon waste streams, and carbon from municipal solid waste. The epoxide reagent and carbon monoxide reagent with high biobased carbon content may have a biobased content of at least 10% and preferably at least 20%, more preferably at least 50%. A biobased content of at least 90%, at least 95%, at least 99%, or 100% is particularly preferred. The epoxide reagents and carbon monoxide reagents with high biobased content may have certain organic impurities. In some embodiments, multifunctional catalysts may include a first portion selective for epoxides and carbon monoxide over other organic impurities.

The processes of the present invention include multifunctional catalysts configured to provide for carbonylation of an epoxide reagent with a carbon monoxide reagent. The multifunctional catalyst has a first portion with a carbonylation functionality. In certain preferred embodiments, the first portion with the carbonylation functionality may comprise a metal carbonyl-Lewis acid. In preferred embodiments, an epoxide reagent and a carbon monoxide reagent may be introduced to a reaction vessel containing a multifunctional catalyst with a first portion having a carbonylation functionality. The epoxide reagent and carbon monoxide reagent may contact and react with the first portion having a carbonylation functionality to produce a beta-lactone intermediate. In some embodiments, the beta-lactone intermediate may react with the first portion having a carbonylation functionality to produce a succinic anhydride intermediate and/or a succinic anhydride product wherein the succinic anhydride intermediate may react with a second portion of the multifunctional catalyst and the succinic anhydride product may be removed from the reaction vessel. In other embodiments, the beta-lactone intermediate may react with a second portion of the multifunctional catalyst having a carbonylation functionality selective to reacting with the beta-lactone intermediate to produce a succinic anhydride intermediate and/or a succinic anhydride product.

The carbonylation functionality of a multifunctional catalyst may utilize a metal carbonyl-Lewis acid moiety such as those described in U.S. Pat. No. 6,852,865. In certain embodiments, the carbonylation functionality includes one or more molecules of the carbonylation catalysts disclosed in U.S. patent application Ser. Nos. 10/820,958; and 10/586,826. In certain embodiments, the carbonylation functionality includes one or more molecules of the catalysts disclosed in U.S. Pat. Nos. 5,310,948; 7,420,064; and 5,359,081. The entirety of each of the preceding references is incorporated herein by reference. In certain preferred embodiments, the metal carbonyl-Lewis acid moiety is bonded or otherwise tethered to a heterogenous support comprising insoluble siliceous material or an insoluble carbon matrix.

Table 1 illustrated below includes Column A directed to a non-exhaustive list of epoxides which may undergo carbonylation to produce beta-lactone intermediates according to the processes of the present invention and Column B directed to a non-exhaustive list of the beta-lactone intermediates.

TABLE 1

| Column A | Column B |
|---|---|
| 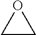 | 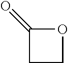 |
| 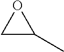 | 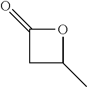 |
| | or/and |
| | 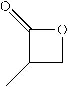 |
| 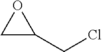 | 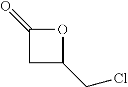 |
| 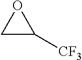 | 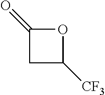 |
| 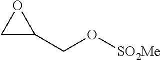 | 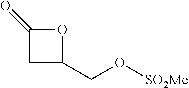 [SMILES: CS(=O)(=O)OCC1CC(=O)O1] |
| 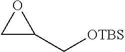 [SMILES: CC(C)(C)[Si](C)(C)OCC1CO1] | 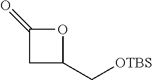 [SMILES: CC(C)(C)[Si](C)(C)OCC1CC(=O)O1] |
| 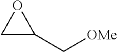 | 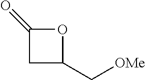 |
| 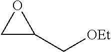 | 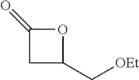 |
| 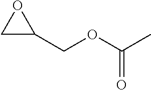 | 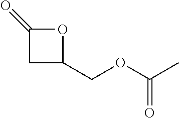 |
| 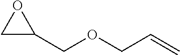 | 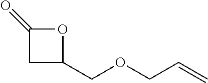 |
| 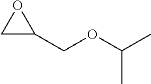 | 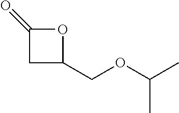 |
| 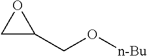 | 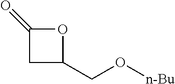 |
| 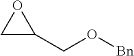 [SMILES: c1ccc(COCC2CO2)cc1] | 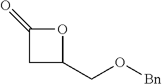 [SMILES: O=C1CC(COCc2ccccc2)O1] |
| 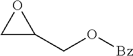 | 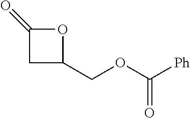 [SMILES: O=C1CC(COC(=O)c2ccccc2)O1] |
| 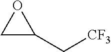 | 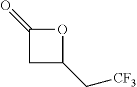 |
| 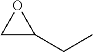 | 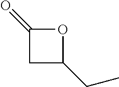 |
| 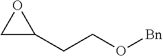 [SMILES: c1ccc(COCCC2CO2)cc1] | 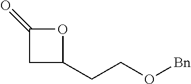 [SMILES: O=C1CC(CCOCc2ccccc2)O1] |
| 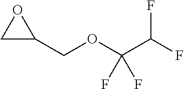 [SMILES: FC(F)C(F)(F)OCC1CO1] | 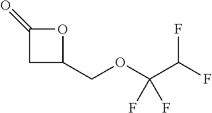 [SMILES: O=C1CC(COC(F)(F)C(F)F)O1] |
| 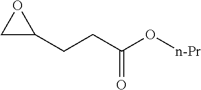 | 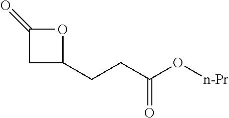 |
| 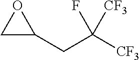 [SMILES: FC(F)(F)C(F)(CC1CO1)C(F)(F)F] | 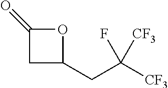 [SMILES: O=C1CC(CC(F)(C(F)(F)F)C(F)(F)F)O1] |
| 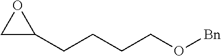 [SMILES: c1ccc(COCCCCC2CO2)cc1] | 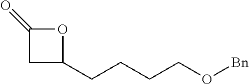 [SMILES: O=C1CC(CCCCOCc2ccccc2)O1] |
| 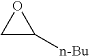 | 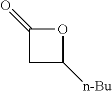 |
| 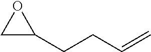 | 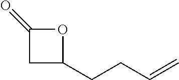 |
| 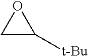 | 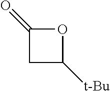 |
| 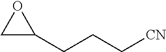 | 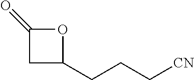 |
| 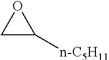 | 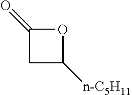 |
| 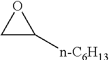 | 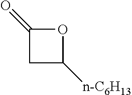 |
| 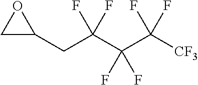 [SMILES: FC(F)(F)C(F)(F)C(F)(F)C(F)(F)CC1CO1] | 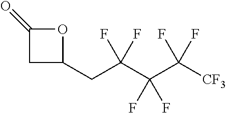 [SMILES: O=C1CC(CC(F)(F)C(F)(F)C(F)(F)C(F)(F)F)O1] |
| 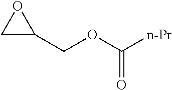 | 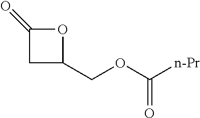 |
| 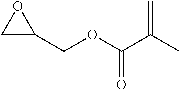 | 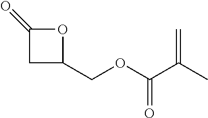 |
| 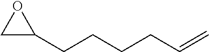 | 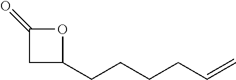 |
| 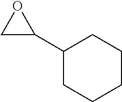 | 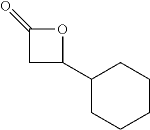 [SMILES: O=C1CC(C2CCCCC2)O1] |
| 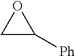 | 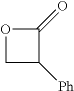 [SMILES: O=C1OCC1c1ccccc1] |
| 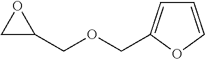 [SMILES: c1coc(COCC2CO2)c1] | 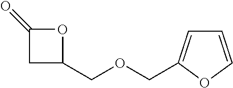 [SMILES: O=C1CC(COCc2ccco2)O1] |
| 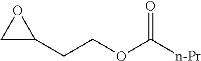 | 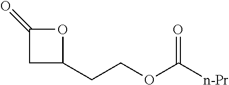 |
| 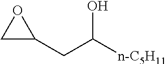 | 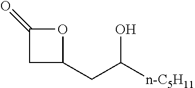 |
| 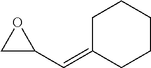 | 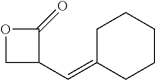 [SMILES: O=C1OCC1C=C1CCCCC1] |
| 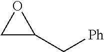 | 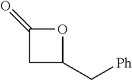 [SMILES: O=C1CC(Cc2ccccc2)O1] |
| 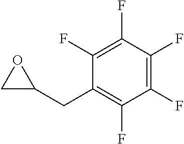 [SMILES: Fc1c(F)c(F)c(CC2CO2)c(F)c1F] | 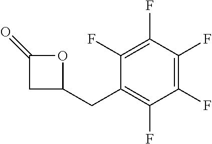 [SMILES: O=C1CC(Cc2c(F)c(F)c(F)c(F)c2F)O1] |
| 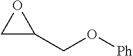 [SMILES: c1ccc(OCC2CO2)cc1] | 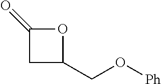 [SMILES: O=C1CC(COc2ccccc2)O1] |
| 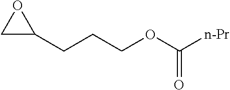 | 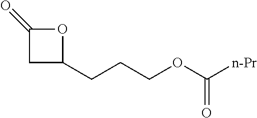 |
| 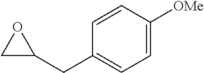 [SMILES: COc1ccc(CC2CO2)cc1] | 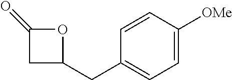 [SMILES: COc1ccc(CC2CC(=O)O2)cc1] |
| 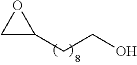 | 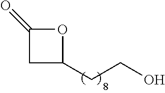 |
| 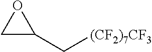 | 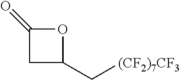 |
| 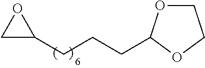 | 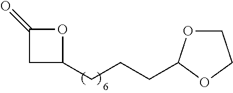 |
| 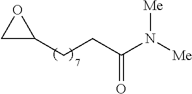 | 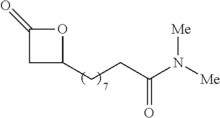 |
| 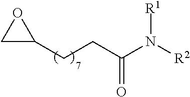 | 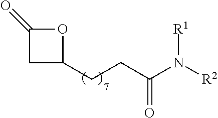 |
| 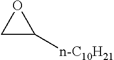 | 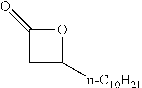 |
| 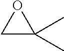 | 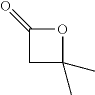 |
| | and/or |
| | 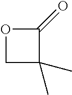 |
| 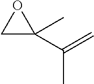 | 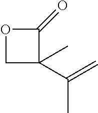 |
| 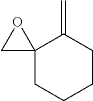 | 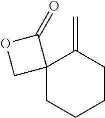 |
| 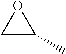 | 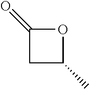 |
| 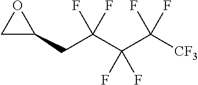 [SMILES: FC(F)(F)C(F)(F)C(F)(F)C(F)(F)C[C@H]1CO1] | 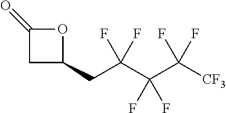 [SMILES: O=C1C[C@H](CC(F)(F)C(F)(F)C(F)(F)C(F)(F)F)O1] |
| 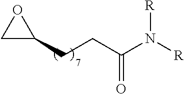 | 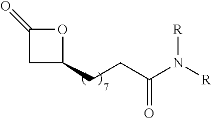 |
| 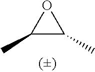 | 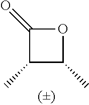 |
| 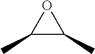 | 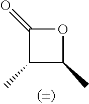 |
| 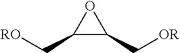 | 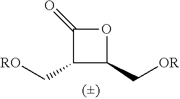 |
| 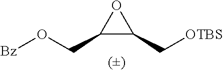 | 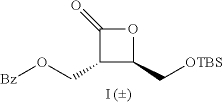 |
| | and/or |
| | 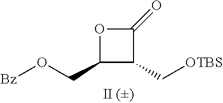 |
| 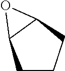 | 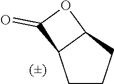 |
| 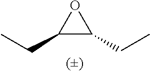 | 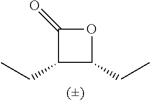 |
| 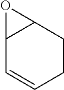 | 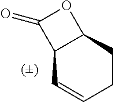 |
| 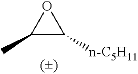 | 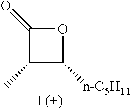 |
| | and/or |
| | 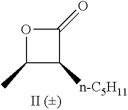 |
| 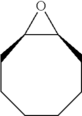 | 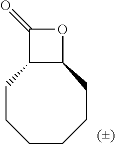 [SMILES: O=C1O[C@H]2CCCCCC[C@H]12] |
| 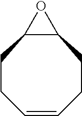 | 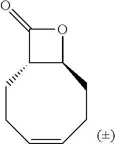 [SMILES: O=C1O[C@H]2CC/C=C\CC[C@H]12] |
| 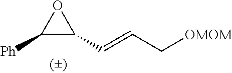 | 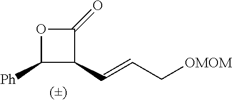 |
| 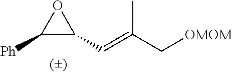 | 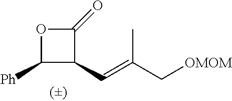 |
| 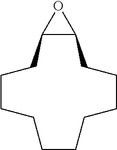 [SMILES: C1CCCCC[C@H]2O[C@H]2CCCC1] | 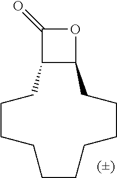 [SMILES: O=C1O[C@H]2CCCCCCCCCC[C@H]12] |
| 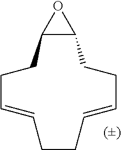 [SMILES: C1=C/CC[C@H]2O[C@@H]2CC/C=C/CC/1] | 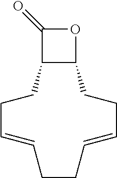 [SMILES: O=C1O[C@@H]2CC/C=C/CC/C=C/CC[C@H]12] |
| | and/or |
| | 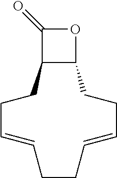 [SMILES: O=C1O[C@@H]2CC/C=C/CC/C=C/CC[C@@H]12] |
| 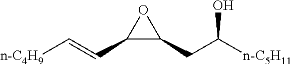 | 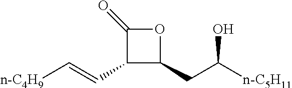 |
| 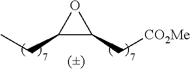 | 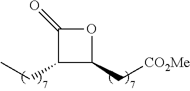 |
| | and/or |
| | 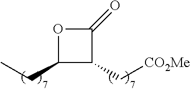 |
| 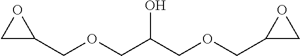 [SMILES: OC(COCC1CO1)COCC1CO1] | 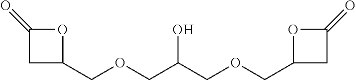 [SMILES: O=C1CC(COCC(O)COCC2CC(=O)O2)O1] |
| 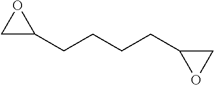 | 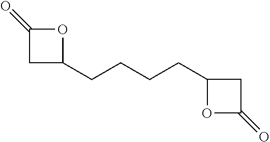 [SMILES: O=C1CC(CCCCC2CC(=O)O2)O1] |
| 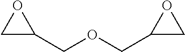 | 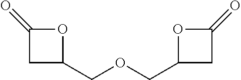 [SMILES: O=C1CC(COCC2CC(=O)O2)O1] |

In preferred embodiments of the present invention, the multifunctional catalysts may include one or more portions having polymerization functionality for producing a polymer. The term “polymer”, as used herein, refers to a molecule of high relative molecular mass, the structure of which comprises the multiple repetitions of units derived, actually or conceptually, from molecules of low relative molecular mass. In certain preferred embodiments, a polymer is comprised of only one monomer species (e.g., an epoxide). In certain other preferred embodiments, a polymer is a copolymer, terpolymer, heteropolymer, block copolymer, or tapered heteropolymer of one or more epoxides.

In preferred embodiments, the processes of the present invention comprise the steps: introducing an epoxide reagent and a carbon monoxide reagent to a reaction vessel; contacting the epoxide reagent and a carbon monoxide reagent with a first portion of a multifunctional catalyst having carbonylation functionality to produce a beta-lactone intermediate; contacting the beta-lactone intermediate with a second portion of the multifunctional catalyst having ring-opening polymerization functionality to produce a polylactone product; and removing the polylactone product from the reaction vessel.

In preferred embodiments, the processes of the present invention provide for ring-opening polymerization. The multifunctional catalyst may have a first portion with a carbonylation functionality and a second portion having ring-opening polymerization functionality. The second portion having ring-opening polymerization functionality may comprise a carboxylate salt of an organic cation, metal-containing complexes, protonated amine, quaternary ammonium salt, guanidinium group, and/or optionally substituted nitrogen-containing heterocycle to name a few. The epoxide reagent and carbon monoxide reagent may be introduced to a reaction vessel and react with the first portion having a carbonylation functionality to produce a beta-lactone intermediate. The beta-lactone may react with the second portion having ring-opening polymerization functionality to produce a polylactone intermediate and/or polylactone product wherein the polylactone intermediate may react with a third portion of the multifunctional catalyst and the polylactone product may be removed from the reaction vessel.

The portions of the multifunctional catalyst suitable for the ring-opening polymerization may include one or more moieties from the following: Journal of the American Chemical Society (2002), 124(51), 15239-15248 Macromolecules, vol. 24, No. 20, pp. 5732-5733, Journal of Polymer Science, Part A-1, vol. 9, No. 10, pp. 2775-2787; Inoue, S., Y. Tomoi, T. Tsuruta & J. Furukawa; Macromolecules, vol. 26, No. 20, pp. 5533-5534; Macromolecules, vol. 23, No. 13, pp. 3206-3212; Polymer Preprints (1999), 40(1), 508-509; Macromolecules, vol. 21, No. 9, pp. 2657-2668; and Journal of Organometallic Chemistry, vol. 341, No. 1-3, pp. 83-9; and in U.S. Pat. Nos. 3,678,069, 3,169,945, 6,133,402; 5,648,452; 6,316,590; 6,538,101; and 6,608,170. The entirety of each of which is hereby incorporated herein by reference. In certain preferred embodiments, a moiety of the portions of the multifunctional catalyst suitable for the ring-opening polymerization may be boned or otherwise tethered to a heterogenous support comprising insoluble siliceous material or an insoluble carbon matrix.

In certain embodiments, polymerization of a beta-lactone intermediate is performed in the presence of polymerization initiator to produce a polylactone product. In some embodiments, the polymerization initiator is an ionic initiator. In variations of this aspect, the ionic initiator has the general formula of M″X where M″ is cationic and X is anionic. M″ is selected from the group consisting of Li+, Na+, K+, Mg2+, Ca2+, and Al3+. In some embodiments, M″ is Na+. In some embodiments, M″ is an organic cation. In some embodiments, the organic cation is selected from the group consisting of quaternary ammonium, imidazolium, and bis(triphenylphosphine)iminium. In some embodiments, the quaternary ammonium cation is tetraalkyl ammonium. In some embodiments, M″ is a metal-containing complex and X is a nucleophilic anion. Suitable nucleophilic anions include, but not limited to, compounds comprising at least one carboxylate group, at least one alkoxide group, at least one phenoxide group, and combination thereof. In some embodiments, the nucleophilic anion is selected from the group consisting of halides, hydroxide, alkoxide, carboxylate, and combination thereof. In some embodiments, the ionic initiator is sodium acrylate. In some embodiments, the ionic initiator is tetrabutylammonium acrylate. In certain preferred embodiments, M″ is bonded or otherwise tethered to a heterogenous support comprising insoluble siliceous material or an insoluble carbon matrix.

In certain preferred embodiments, the processes of the present invention provide for the carbonylation of an epoxide reagent with a carbon monoxide reagent to produce a beta-lactone intermediate and for the subsequent co-polymerization of the beta-lactone intermediate with another reagent. In certain preferred embodiments, the processes include a multifunctional catalyst with a first portion having carbonylation functionality and at least a second portion with co-polymerization functionality. In certain embodiments, the beta-lactone intermediate may be co-polymerized with monomers or another polymer having hydroxyl functional groups such as simple alcohols, diols, triols, polyols, and sugar alcohols.

In certain preferred embodiments, the beta-lactone intermediate may be co-polymerized with a cyclic anhydride such as a succinic anhydride intermediate. The co-polymerization of a beta-lactone intermediate and a cyclic anhydride may be performed in the presence of polymerization initiator. In certain preferred embodiments, the processes of the present invention provide for co-polymerization. The multifunctional catalyst may have a first portion with a carbonylation functionality and one or more other portions with co-polymerization functionality. The one or more other portions may comprise condensation polymerization catalysts, oxide catalysts, precious metal catalysts, and/or combinations thereof. In preferred embodiments providing for co-polymerization, one or more reagents may be introduced to the reaction vessel for reaction with a beta-lactone intermediate. For example, a cyclic anhydride reagent may be introduced to the reaction vessel for co-polymerization with a beta-lactone intermediate.

In certain preferred embodiments, an epoxide reagent and carbon monoxide reagent may contact and react with a first portion of a multifunctional catalyst having a carbonylation functionality to produce a beta-lactone intermediate; the beta-lactone intermediate may partially react with the first portion of a multifunctional catalyst having a carbonylation functionality to produce a succinic anhydride intermediate; and the beta-lactone intermediate and succinic anhydride intermediate may contact a second portion of the multifunctional catalyst having co-polymerization functionality to produce a co-polymer product. In certain embodiments, the amount of succinic anhydride intermediate formed may be controlled by introducing a stochiometric excess of the carbon monoxide reagent. In certain embodiments, co-polymerization may be controlled by the introduction of a polymerization initiator, the physical location of the portion having co-polymerization functionality, and/or the stochiometric ratio of the beta-lactone intermediate to the cyclic anhydride.

In preferred embodiments, the processes of the present invention provide for carbonylation to produce a beta-lactone intermediate and for subsequent modification of the beta-lactone intermediate. The processes may include a multifunctional catalyst comprising at least one metal complex having at least one ligand with a cationic moiety of at least two, at least one anionic metal carbonyl compound, and at least one anionic nucleophile. The multifunctional catalyst having at least one metal complex may catalyze carbonylation of an epoxide in the presence of carbon monoxide to form a beta-lactone intermediate and subsequently catalyze modification the beta-lactone intermediate with an alcohol reagent and/or acid reagent. In certain embodiments, the multifunctional catalyst may catalyze carbonylation of an epoxide in the presence of carbon monoxide to form a beta-lactone intermediate and catalyze the subsequent polymerization of the beta-lactones to provide polylactone oligomers. In some embodiments, the multifunctional catalyst may catalyze subsequent copolymerization of the beta-lactones and/or polylactone oligomers with polyols.

In certain preferred embodiments, the processes of the present invention may provide for modification of a beta-lactone with an ammonia reagent to provide an acrylonitrile product. In certain embodiments, a multifunctional catalyst has a first portion with carbonylation functionality and a second portion comprising a dehydration agent chosen from the following: a phosphorous pentoxide, an organophosphorous compound, a carbodiimide compound, a triazine compound, an organosilicon compound, a transition metal complex, an aluminum complex, or a mixture thereof.

In certain preferred embodiments, the portions of the multifunctional catalyst suitable for modification of a beta-lactone intermediate may include metal, metal oxides, metal nitrides wherein the metal may be chosen from the group including Cr, Al, V, Mn, Fe, Mo, Sn, Bi, As, Sb, Si, Ce, Zn, Zr, and U. In certain preferred embodiments, the portion is supported such as on silica. Certain examples of a portion of the multifunctional catalyst with modification functionality useful for modification of a beta-lactone intermediate include Cr2O3/Al2O3, KNaMoP/Al2O3, NaMo/Al2O3, AsFeO, SbSnO, FeBiPO, BiMoO, MoO3, MoO3/SiO2, and/or NaMo/Al2O3. In certain embodiments, a portion of the heterogenous catalyst with modification functionality may include a phosphorous pentoxide, an organophosphorous compound, a carbodiimide compound, a triazine compound, an organosilicon compound, a transition metal complex, an aluminum complex, or a mixture thereof.

In certain preferred embodiments, the multifunctional catalysts of the present invention may be formed in one reaction vessel. In some embodiments, the catalysts of the present invention may be formed from catalyst reagents dissolved in solution such as a THF solvent or hexane solvent. In some embodiments, the catalysts of the present invention may be formed by pyrolyzing catalyst reagents in a reaction vessel. In some embodiments, the formation of the catalysts and/or catalyst reagents may include the chemical or electrochemical reduction of metal halides. In some embodiments, metal, alloy, metal alkyls, metal hydrides, complex metal hydrides, metal anhydrides, or mixtures may be reducing agents. In some embodiments, hydrogen or carbon monoxide may be used as a reducing agent. In some embodiments, a reducing agent may contain a sulfur atom.

In preferred embodiments, the multifunctional catalysts of the present invention may include at least one metal complex with the formula [(Lc)aMb]z+. The Lc is the ligand that includes at least two cationic moieties. If two or more Lc are present, then each may be the same or different. The M is a metal atom. If two or more M are present, then each may be the same or different. The a is an integer from 1 to 4 inclusive; the b is an integer from 1 to 2 inclusive; and the z is an integer greater than 2 that represents the cationic charge on the metal complex. The metal atom may be chosen from titanium, chromium, manganese, iron, ruthenium, cobalt, rhodium, samarium, rhenium, iridium, zirconium, nickel, palladium, copper, zinc, magnesium, aluminum, gallium, tin, indium, molybdenum, and tungsten.

In preferred embodiments, the multifunctional catalyst may comprise a metal complex having at least one tetradentate ligand such as porphyrin. In certain preferred embodiments, the tetradentate ligand may be a salen ligand. In certain embodiments, the salen ligand may include salicylaldehyde and ethylenediamine molecular parts. In certain embodiments, the metal complex includes a cobalt atom. In certain embodiments, the metal complex may be reacted with divinylbenzene to form an insoluble resin which may function as a heterogenous catalyst.

In certain preferred embodiments, the multifunctional catalyst comprises at least two metal complexes wherein each metal complex includes a metal atom chosen from the group: titanium, chromium, manganese, iron, ruthenium, cobalt, rhodium, samarium, rhenium, iridium, zirconium, nickel, palladium, copper, zinc, magnesium, aluminum, gallium, tin, indium, molybdenum, and tungsten. In certain preferred embodiments, the multifunctional catalyst comprising at least two metal complexes may further comprise a first metal complex having a different concentration than a second metal complex based on the total weight of the multifunctional catalyst. In some embodiments, the second metal complex may be present in an amount from 0.1 to 99 wt. %, e.g., from 0.1 to 95 wt. %, or from 0.1 to 90 wt. %. In some embodiments, the second metal complex may be present in the multifunctional catalyst in an amount from 0.1 to 90 wt. %, e.g. from 0.1 to 80 wt. %, or from 0.1 to 75 wt. %. In some embodiments, the second metal complex may be present in an amount from 0.1 to 75 wt. %, e.g., from 0.1 to 70 wt. %, or from 0.1 to 50 wt. %. In some embodiments, the second metal complex may be present in an amount from 0.1 to 50 wt. %, e.g., from 0.1 to 25 wt. %, or from 0.1 to 10 wt. %. In some embodiments, the second metal complex may be present in an amount from 0.1 to 10 wt. %, e.g., from 0.1 to 5 wt. % or from 0.1 to 1 wt. %.

In certain preferred embodiments of the present invention, the multifunctional catalyst includes at least two metal complexes and one or more bridging ligands. The at least two metal complexes further comprises a first metal complex and a second metal complex. A portion of the one or more bridging ligands may be covalently or coordinately bonded to the first metal complex and the second metal complex for tethering the first metal complex to the second metal complex. Bridging ligands may comprise C1-20 aliphatic groups and/or C1-20 heteroaliphatic having 1-10 heteroatoms independently selected from the group consisting of nitrogen, boron, oxygen, and sulfur. In some embodiments, the bridging ligands comprise 6- to 10-membered aryl group and/or 5- to 10-membered heteroaryl having 1-4 heteroatoms independently selected from nitrogen, oxygen, and sulfur. In some embodiment, the bridging ligands comprise 4- to 7-membered heterocyclic having 1-2 heteroatoms independently selected from the group consisting of nitrogen, boron, oxygen, and sulfur. In certain embodiments, bridging ligands provide for close proximity between two portions of the multifunctional catalysts having different functionalities.

Embodiments of the present invention may include one or more portions of the multifunctional catalyst that are bonded or otherwise tethered to a heterogenous support. In certain embodiments, the heterogenous support comprises a siliceous based material, e.g., silica, and/or a carbon based material, e.g., carbon black or activated carbon, although any of a variety of other suitable supporting materials may be used. The heterogenous support material may be selected from the group comprising silica, magnesia, alumina, titania, silica/alumina, pyrogenic silica, high purity silica, zirconia, zincate, carbon (e.g., carbon black or activated carbon), zeolites and mixtures thereof. In certain embodiments, the heterogenous support comprises a polymer. In certain embodiments, the heterogenous support includes one or more metal complexes. Preferably, the heterogenous support comprises a siliceous material such as silica, pyrogenic silica, or high purity silica. In some embodiments, the heterogenous support siliceous material is substantially free of alkaline earth metals, such as magnesium and calcium. In some embodiments, the heterogenous support is present in an amount from about 25 wt. % to about 99 wt. %, e.g., from about 30 wt. % to about 98 wt. % or from about 35 wt. % to about 95 wt. %, based on the total weight of the multifunctional catalyst.

In preferred embodiments, the heterogenous support comprises a siliceous material, e.g., silica, having a surface area of at least about 50 m2/g, e.g., at least about 100 m2/g, or at least about 150 m2/g. In terms of ranges, the siliceous support material preferably has a surface area from about 50 to about 800 m2/g, e.g., from about 100 to about 500 m2/g or from about 100 to about 300 m2/g. High surface area silica, as used throughout the application, refers to silica having a surface area of at least about 250 m2/g. For purposes of the present specification, surface area refers to BET nitrogen surface area, meaning the surface area as determined by ASTM D6556-04, the entirety of which is incorporated herein by reference.

The preferred siliceous material also preferably has an average pore diameter from about 5 to about 100 nm, e.g., from about 5 to about 50 nm, from about 5 to about 25 nm or from about 5 to about 10 nm, as determined by mercury intrusion porosimetry, and an average pore volume from about 0.5 to about 3.0 cm3/g, e.g., from about 0.7 to about 2 cm3/g or from about 0.8 to about 1.5 cm3/g, as determined by mercury intrusion porosimetry.

In some embodiments, the morphology of the support material and/or of the catalyst composition may be pellets, extrudates, spheres, spray dried microspheres, rings, pentarings, trilobes, quadrilobes, multi-lobal shapes, or flakes although cylindrical pellets are preferred. Preferably, the siliceous support material has a morphology that allows for a packing density from about 0.1 to about 2.0 g/cm3, e.g., from about 0.2 to about 1.5 g/cm3 or from about 0.3 to about 0.5 g/cm3.

In some embodiments, one or more portions of the multifunctional catalyst may be impregnated into the heterogenous support. With impregnation, a moiety of at least one portion and heterogenous support material are mixed together followed by drying and calcination to form the final multifunctional catalyst with heterogenous support. With simultaneous impregnation, it may be desired to employ a dispersion agent, surfactant, or solubilizing agent, e.g., ammonium oxalate or an acid such as acetic or nitric acid, to facilitate the dispersing or solubilizing of the first, second and/or optional third metal complex in the event one or more of the metal complexes are incompatible.

With sequential impregnation, a first portion may be first added to the heterogenous support material followed by drying and calcining, and the resulting material may then be impregnated by subsequent one or more portions followed by an additional drying and calcining to form the final multifunctional catalyst with heterogenous support. In some embodiments, additional portions may be added either with the first and/or second portion or in a separate sequential impregnation, followed by drying and calcination. In some embodiments, combinations of sequential and simultaneous impregnation may be employed if desired.

In preferred embodiments, the multifunctional catalyst may be employed in a fixed bed reactor where the reactants are passed over or through the catalyst. Other reactors, such as fluid or ebullient bed reactors, can be employed. In some instances, the multifunctional catalysts may be used in conjunction with an inert material to regulate the pressure drop of the reactant stream through the catalyst bed and the contact time of the reactant compounds with the multifunctional catalyst.

The multifunctional catalyst may be presented within a single-phase or multiphase metal oxide catalysts. The quintessential example of a single-phase, heterogeneous, selective-oxidation catalyst is bismuth molybdate. An incorporation of catalytically-functional, elemental components, such as a solid solution with a catalytically-active, scheelite bismuth molybdate phase is first demonstrated with iron and a single-phase, mixed-metal oxide with the formulation Bi3FeMo2O12. Physical properties of mixed-metal oxides are important component of their multifunctional character and equally critical to their ability to function fully and effectively in the arduous environment of industrial operations.

### EXAMPLE 1

### Conversion from Ethylene Oxide to Beta-Propiolactone as an Intermediate and Acrylic Nitrile by using Multifunctional Catalysts

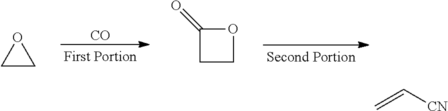

A 300 mL Parr reactor is dried overnight under vacuum. In a nitrogen glovebox, the reactor is charged with [(CITPP)Al][Co(CO)4] (66 mg, 60 mmol) on a first portion of a solid support, such as Zeolite Y hydrogen (10 g), hexamethylbenzene (162 mg, 1.0 mmol), and THF (dried over 4 Å molecular sieves, and freeze, pump, and thaw 3 times). The reactor is then closed and removed from the glovebox. Zeolite Y hydrogen (80:1 mole ratio SiO2/Al2O3, powder S.A. 780 m2/g) is dried under vacuum at 100° C. for one day before use. Ethylene oxide is vacuum transferred to a transfer vessel from EO lecture bottle. The Parr reactor is cooled to −78° C. and high vacuum is applied to the reactor. The vacuum is disconnected from the reactor, and the transfer vessel is connected to the Parr reactor to allow EO to be vacuum transferred from the transfer vessel to the reactor at −78° C. The reaction mixture is warmed to ambient temperature and saturated with CO by pressurizing the reactor with CO to three fourths of the desired CO pressure (e.g. 150 psi). The reaction mixture is then heated to the desired temperature. After the reaction mixture reaches the desired temperature, the reactor is pressurized to the desired pressure (e.g. 200 psi). The reaction mixture is agitated for 3 h. The reactor is cooled to <0° C. and vented. A portion of the reaction mixture is sampled and analyzed by 1H NMR in CDCl3.

Phenothiazine is the polymerization inhibitor used. Phenothiazine (9.0 mg) is added using a needle valve to a mixture of sulfolane (40.0 g), beta-propiolactone, and Zeolite Y hydrogen (20.0 g) at 165° C. with 50 psi of carbon monoxide. The reaction mixture is heated to 170° C. to produce acrylic acid.

The 300 mL Parr reactor is purged and filled with NH3 until the pressure reached 0.75 MPa. Then O2 is introduced until the total pressure reaches 1.25 MPa. The reaction mixture is stirred at a controlled temperature (400° C. for 2 h). The intermediate acrylic acid is reacted with NH3 under oxygen and catalyst of MoO3/γ-Al2O3 on the second portion of Zeolite Y hydrogen to form acrylic nitrile. After the reaction, the mixture is filtered. The filtrate is analyzed by GC-MS and GC using acrylonitrile as an internal standard. For recycling tests, the catalyst is filtered after the reaction, washed with acetone three times, and then washed with doubly distilled water several times. Then, it is dried at 110° C., calcined at 400° C. for 4 h, and then used for the next run.

### EXAMPLE 2

### Conversion from Epoxides to Succinic Anhydride as an Intermediate and Poly(Propylene Succinate) by Using Multifunctional Catalysts

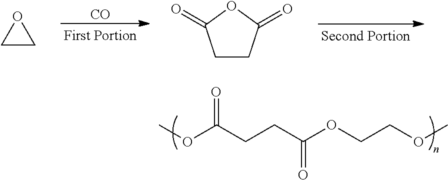

A 300 mL Parr reactor is dried overnight under vacuum. In a nitrogen glovebox, the reactor is charged with [(CITPP)Al][Co(CO)4] (66 mg, 60 mmol) on a first portion of a solid support, such as Zeolite Y hydrogen (10 g), hexamethylbenzene (162 mg, 1.0 mmol), and THF (dried over 4 Å molecular sieves, and freeze, pump, and thaw 3 times). The reactor is then closed and removed from the glovebox. Ethylene oxide is vacuum transferred to a transfer vessel from EO lecture bottle. The Parr reactor is cooled to −78° C. and high vacuum is applied to the reactor. The vacuum is disconnected from the reactor, and the transfer vessel is connected to the Parr reactor to allow EO to be vacuum transferred from the transfer vessel to the reactor at −78° C. The reaction mixture is warmed to ambient temperature and saturated with CO by pressurizing the reactor with CO to three fourths of the desired CO pressure (e.g. 150 psi), then heated to the desired temperature. After the reaction mixture reaches the desired temperature, the reactor is pressurized to the desired pressure (e.g. 200 psi). The reaction mixture is agitated for 3 h. The reactor is cooled to <0° C. and vented. A portion of reaction mixture is sampled and analyzed by 1H NMR in CDCl3.

Ethylene oxide is vacuum transferred from the transfer vessel to the 300 mL Parr reactor containing 0.01 mole of [Cl-salcy)CoNO3], [PPN][NO3] as a polymerization initiator/catalyst on the second portion of a solid support, such as Zeolite Y hydrogen (10 g) in 540 ml of 1,2-dimethoxyethane. The mixture is stirred at 30° C., for 36 hours. After cooling to room temperature, the prepolymer is filtered, washed with ether, and dried. The yield is 8.5 g, and corresponding to a molecular weight of 8600. The molecular weight determined with the aid of gel permeation chromatography is 400,000.

### EXAMPLE 3

### Conversion from Ethylene Oxide to Beta-Propiolactone and Acrylic Nitrile as an Intermediate and Polyacrylonitrile as a Product by Using Multifunctional Catalysts

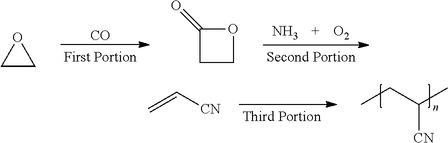

A 300 mL Parr reactor is dried overnight under vacuum. In a nitrogen glovebox, the reactor is charged with [(CITPP)Al][Co(CO)4] (66 mg, 60 mmol) on a first portion of a solid support, such as Zeolite Y hydrogen (10 g), hexamethylbenzene (162 mg, 1.0 mmol), and THF (dried over 4 Å molecular sieves, and freeze, pump, and thaw 3 times). The reactor is then closed and removed from the glovebox. Zeolite Y hydrogen (80:1 mole ratio SiO2/Al2O3, powder S.A. 780 m2/g) is dried under vacuum at 100° C. for one day before use. Ethylene oxide is vacuum transferred to a transfer vessel from EO lecture bottle. The Parr reactor is cooled to −78° C. and high vacuum is applied to the reactor. The vacuum is disconnected from the reactor, and the transfer vessel is connected to the Parr reactor to allow EO to be vacuum transferred from the transfer vessel to the reactor at −78° C. The reaction mixture is warmed to ambient temperature and saturated with CO by pressurizing the reactor with CO to three fourths of the desired CO pressure (e.g. 150 psi). The reaction mixture is then heated to the desired temperature. After the reaction mixture reaches the desired temperature, the reactor is pressurized to the desired pressure (e.g. 200 psi). The reaction mixture is agitated for 3 h. The reactor is cooled to <0° C. and vented. A portion of reaction mixture is sampled and analyzed by 1H NMR in CDCl3.

Phenothiazine is the polymerization inhibitor used. Phenothiazine (9.0 mg) is added using a needle valve to a mixture of sulfolane (40.0g), beta-propiolactone, and Zeolite Y hydrogen (20.0 g) at 165° C. with 50 psi of carbon monoxide. The reaction mixture is heated to 170° C. to produce acrylic acid.

The 300 mL Parr reactor is purged and filled with NH3 until the pressure reaches 0.75 MPa. Then O2 is introduced until the total pressure reaches 1.25 MPa. The reaction mixture is stirred at a controlled temperature (400° C. for 2 h). The intermediate acrylic acid is reacted with NH3 under oxygen and a catalyst of MoO3/γ-Al2O3 on the second portion of Zeolite Y hydrogen to form acrylic nitrile. After the reaction, the mixture is filtered. The filtrate is analyzed by GC-MS and GC using acrylonitrile as an internal standard. For recycling tests, the catalyst is filtered after the reaction, washed with acetone three times, and then washed with doubly distilled water several times. Then, it is dried at 110° C., calcined at 400° C. for 4 h, and then used for the next run.

Startup is achieved by charging the 300 mL Parr reactor having agitation means therein with approximately, 200 parts of monomer mixture and 3800 parts of water. The reaction temperature is brought to 45 to 50° C. Acrylonitrile is initiated and the reaction maintains and is catalyzed by zinc formaldehyde sulfoxylate on a third portion of the catalyst within the noted temperature range by thermostatic control. Zinc formaldehyde sulfoxylate is bonded to a heterogenous support. Polymer containing aqueous effluent is continuously drawn from the reactor to maintain a relatively constant fluid level in the reactor. A reaction residence time of approximately 4.5 hours results in the establishment of equilibrium conditions for continuous steady-state production. At steady-state conditions, the reaction media contain a catalyst-initiator concentration of approximately 0.5 percent potassium persulfate, 1.58 percent sodium metabisulfite, 0.65 percent sodium acetate and a trace of ferrous sulfate. Sufficient sulfuric acid is added to maintain a pH of about 3 to 4 in the reaction media. At steady-state conditions, the reaction media comprise approximately 24 percent monomer polymer and 76 percent water. Dilute water solutions of catalysts and initiator are fed into the reactor at the rate of 1,862 parts per hour, while solutions of monomer to provide the desired weight ratio in the produced polymer are fed to the reactor at a rate of 1,138 parts per hour. Polymer containing effluent is removed from the reactor at a rate of 3,000 parts per hour, which provides an average residence time of 90 minutes in the reactor.

The reactor effluent is cooled to approximately ambient temperature. The polymer is filtered and the aqueous effluent returns to the reactor along with replenishing amounts of reactants.

The filter cake is washed with water and subsequently passed in wet form to a pressure vessel for solvation with acetonitrile.

### EXAMPLE 4

### Conversion from Epoxides to Betalactones as an Intermediate and Polylactones as Products by Using Multifunctional Catalysts

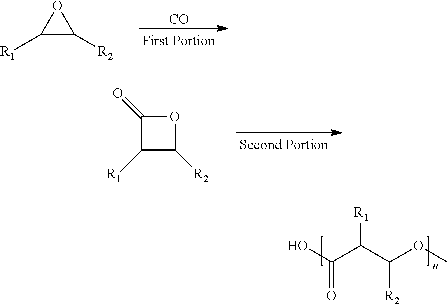

A 100 ml Parr reactor is dried at 90° C., under vacuum overnight. In a drybox, it is cooled in a −35° C. freezer for at least 1.5 hours and equipped with a small test-tube and magnetic stir bar. The test-tube is charged with 0.500 ml of epoxide, stored at −35° C., and charged with [(CITPP)Al][Co(CO)4] on a first portion of a solid support, such as ZSM-5 (MFI) (10 g). Upon removal from the drybox, the reactor is pressurized to a pressure of 900 psi, placed in a preheated oil bath and the reactor is stirred at about 60° C. for about 4-10 hours. When the reaction time has passed, the reactor is cooled in a bath of dry ice/acetone until the pressure reaches a minimum and is then slowly vented. The intermediate mixture is subjected to NMR analysis. The trapping of vented gases indicates that only 2-5% of the material is lost. Vented gases contain the same ratios of compounds (within 3-4%) that remain in the reactor.

0.01 mole of tetrabutylammonium acrylate is used as a polymerization initiator on the second portion of a solid support, such as ZSM-5 (MFI). 540 ml of 1,2-dimethoxyethane is added to the Parr reactor. The mixture is refluxed for 134 hours. After cooling to room temperature, the prepolymer is filtered, washed with ether, and dried. The yield is 8.5 g., corresponding to a molecular weight of 600,000. The molecular weight determined with the aid of gel permeation chromatography is 600,000.

What is claimed is:

1. A multifunctional catalyst, comprising:
a substrate;
a multifunctional catalyst supported on the substrate,
wherein a first portion of the multifunctional catalyst has carbonylation functionality with at least one epoxide reagent and at least one carbon monoxide reagent to produce one or more intermediates; and
wherein a second portion of the multifunctional catalyst has polymerization functionality to polymerize one or more intermediates to form a polymer.

2. The multifunctional catalyst of claim 1, wherein the carbonylation functionality of the multifunctional catalyst includes a carbonylation catalyst wherein the carbonylation catalyst comprises a metal carbonyl-Lewis acid catalyst.

3. The multifunctional catalyst of claim 1, wherein the substrate comprises a heterogeneous support.

4. The multifunctional catalyst of claim 1, wherein the second portion of the multifunctional catalyst includes a moiety comprising an ionic initiator.

5. The multifunctional catalyst of claim 4, wherein the carbonylation functionality of the multifunctional catalyst and the ionic initiator form a mixture on the substrate.

6. The multifunctional catalyst of claim 4, wherein the substrate has a first zone and a second zone downstream of the first zone, wherein at least about 90 weight percent of the carbonylation functionality of the multifunctional catalyst is located in the first zone and at least about 90 weight percent of the polymerization functionality is located in the second zone.

7. The multifunctional catalyst of claim 6, wherein a volume ratio between the first and second zones is from about 1:10 to about 10:1.

8. The multifunctional catalyst of claim 3, wherein the heterogeneous support is selected from the group comprising silica, magnesia, alumina, titania, silica/alumina, pyrogenic silica, high purity silica, zirconia, zincate, carbon, zeolites and mixtures thereof.

9. The multifunctional catalyst of claim 1, wherein the at least one epoxide reagent comprises an ethylene oxide reagent.

10. The multifunctional catalyst of claim 1, wherein the one or more intermediates comprises a beta-lactone intermediate.

11. The multifunctional catalyst of claim 1, wherein the one or more intermediates comprises a succinic anhydride intermediate.

12. A multifunctional catalyst, comprising:
a substrate;
a catalyst supported on the substrate, wherein the catalyst has a first portion of carbonylation functionality designed to catalyze at least one epoxide reagent and at least one carbon monoxide reagent to produce one or more first intermediates; and
a second portion of the catalyst designed to convert the one or more first intermediates to a product or a second intermediate.

13. The multifunctional catalyst of claim 12 further comprising a third portion of polymerization functionality designed to polymerize acrylic nitrile.

14. The multifunctional catalyst of claim 13, wherein the third portion comprises a metal complex bonded to the substrate.

15. The multifunctional catalyst of claim 14, wherein the metal complex comprises a metal chosen from Ti, Cr, Mn, Fe, Ru, Co, Rh, Sm, Re, Ir, Zr, Ni, Pd, Co, Zn, Mg, Al, Ga, Sn, In, Mo, W.

16. The multifunctional catalyst of claim 12, wherein the second portion of the catalyst includes a moiety comprising a dehydration agent.

17. The multifunctional catalyst of claim 16, wherein the dehydration agent is chosen from the group consisting of a phosphorous pentoxide, an organophosphorous compound, a carbodiimide compound, a triazine compound, an organosilicon compound, a transition metal complex, an aluminum complex, and a mixture thereof.

18. The multifunctional catalyst of claim 12, wherein the first portion of the carbonylation functionality includes a moiety comprising a carbonylation catalyst wherein the carboxylation catalyst comprises a metal carbonyl-Lewis acid catalyst.

19. The multifunctional catalyst of claim 12, wherein the substrate comprises a heterogeneous support.

20. The multifunctional catalyst of claim 18, wherein the second portion of the multifunctional catalyst includes a moiety comprising a polymerization initiator.

21. The multifunctional catalyst of claim 20, wherein the polymerization initiator comprises an ionic initiator.

22. The multifunctional catalyst of claim 20, wherein the carbonylation catalyst and the polymerization initiator form a mixture on the substrate.

23. The multifunctional catalyst of claim 20, wherein the substrate has a first zone and a second zone downstream of the first zone, wherein at least 90 weight percent of the carbonylation catalyst is located in the first zone and at least about 90 weight percent of the polymerization initiator is located in the second zone.

24. The multifunctional catalyst of claim 23, wherein a volume ratio between the first and second zones is from about 1:10 to about 10:1.

25. The multifunctional catalyst of claim 19, wherein the heterogeneous support is selected from the group comprising silica, magnesia, alumina, titania, silica/alumina, pyrogenic silica, high purity silica, zirconia, zincate, carbon, zeolites and mixtures thereof.

26. The multifunctional catalyst of claim 12, wherein the at least one epoxide reagent includes an ethylene oxide reagent.

27. The multifunctional catalyst of claim 12, wherein the one or more first intermediates includes a beta-lactone intermediate.

28. The multifunctional catalyst of claim 12, wherein the one or more first intermediates includes a succinic anhydride intermediate.

29. The multifunctional catalyst of claim 12, wherein the second intermediate comprises acrylic acid.
